SEQUENCE LISTING (2 sequences)

SEQ ID NO: 1 - DNA, 19 bp, ARTIFICIAL SEQUENCE
  PRIMER
aagttcatctgcaccaccg

SEQ ID NO: 2 - DNA, 20 bp, ARTIFICIAL SEQUENCE
  PRIMER
tgttctgctggtagtggtcg

(19) United States
(12) Patent Application Publication
(10) Pub. No.: US 2004/0208847 A1
(43) Pub. Date: Oct. 21, 2004

(54) Method and vectors for selectively transducing retinal pigment epithelium cells

(76) Inventors: Fabienne Rolling, Nantes (FR); Michel Weber, Vertou (FR)

Correspondence Address: YOUNG & THOMPSON, 745 SOUTH 23RD STREET 2ND FLOOR, ARLINGTON, VA, 22202

(21) Appl. No.: 10/400,531

(22) Filed: Mar. 28, 2003

(51) Int. Cl. A01N 63/00; A01N 65/00; A61K 48/00; C12N 15/00; C12N 15/09; C12N 15/63; C12N 15/70; C12N 15/74

(52) U.S. Cl. 424/93.2; 424/93.1; 424/93.6; 435/320.1

(57) ABSTRACT

A method for selectively transducing retinal pigment epithelium (RPE) cells in an eye of a mammal, comprises administering to the mammal a vector particle exhibiting an AAV-4 capsid protein.

### FIELD OF THE INVENTION

The present invention relates to the field of gene transfer into the eye, for example in view of treating, preventing or alleviating the effects of a disease in the eye of a mammal. More particularly, the invention concerns the use of a capsid protein from the Adeno-Associated Virus serotype 4 (AAV-4), to specifically target retinal pigment epithelial (RPE) cells. The invention also pertains to compositions and methods for preventing or treating diseases of the eye, using any vector exhibiting a capsid protein from AAV-4 to transfer selected genes suitable for preventing or treating said diseases.

### BACKGROUND AND PRIOR ART

Recombinant AAV-2 vectors are capable of efficient and prolonged transgene expression in a number of tissues and have been used to deliver therapeutic genes to correct defects in animal models of various human disorders.

More recently, seven other rAAV serotypes (AAV-1, 3 , 4, 5, 6, 7, and 8) have been isolated and cloned. A number of in vivo studies showed that these new serotypes displayed tissue preference and, therefore, improved safety. However, so far, none of the new serotypes was reported to exhibit a cell type restriction in a given organ with a conserved tropism among mammalians including a nonhuman primate.

In the retina, following subretinal delivery, AAV-2 vectors transduced retinal pigmented epithelium and photoreceptor cells (Ali, Reichel et al. 1996; Ali, Reichel et al. 1998; Bennett, Maguire et al. 1999), and was successful in delivering ribozymes, photoreceptor genes, and neurotrophic factors in mice and rat models of retinal degeneration (Ali, Sarra et al. 2000; Lau, McGee et al. 2000; LaVail, Yasumura et al. 2000; Green, Rendahl et al. 2001; Liang, Dejneka et al. 2001). Visual function was restored in a canine model of childhood blindness using a rAAV-2 carrying a wtRPE65 gene providing critical preclinical data supporting these vectors for human applications (Acland, Aguirre et al. 2001; Acland et al., WO 02/082904). rAAV chimeric serotypes wherein the vector is flanked by AAV-2 ITRs but encapsidated in an AAV-1, 2, 3, 4 or -5 shell have been studied (Auricchio, Kobinger et al. 2001; Rabinowitz, Rolling et al. 2002; Yang, Schmidt et al. 2002). It was shown that their subretinal delivery resulted in a quantitative transgene expression hierarchy with rAAV-4 and -5 capsids being the most efficient.

### SUMMARY OF THE INVENTION

The inventors have now demonstrated that a recombinant AAV of serotype 4 delivered in the subretinal space of a non human primate leads to exclusive transduction of retinal pigment epithelial (RPE) cells. Since the primate eye is anatomically very similar to the human eye, rAAV-mediated gene transfer in the eye of non human primates is highly relevant with respect to future clinical development in humans. Also, surgical procedures for vector delivery are similar. Therefore, studying vector shedding in this context provides additional important preclinical information.

The present invention hence pertains to a method for selectively transducing retinal pigment epithelium (RPE) cells in an eye of a mammal, comprising administering to said mammal a vector particle exhibiting an AAV-4 capsid protein.

Another object of this invention is to provide a method for preventing, treating or alleviating an eye disease in a mammal, by delivering into the eye of said mammal a vector particle exhibiting an AAV-4 capsid protein and comprising a vector genome encoding a transgene which, when expressed in RPE cells, has a beneficial effect on said eye disease.

A vector particle which exhibits an AAV-4 capsid protein, and comprises a vector genome encoding a transgene which, when specifically expressed in retinal pigment epithelium (RPE) cells, can treat, prevent or alleviate the effects of an eye disease, is also part of the present invention, as well as a pharmaceutical composition for administration in the eye, comprising such vector particles and a pharmaceutically acceptable carrier.

### BRIEF DESCRIPTION OF THE DRAWINGS

FIG. 1. Rat model: Rats were injected with rAAV-2/4. CMV.gfp and analyzed 30 days post injection (p.i.). Fluorescent retinal imaging (A). Sclera/choroid/RPE (B) and neuroretina (C) flatmounts. Sections from sclera/choroid/RPE (D) and neuroretina (E) examined under an inverted fluorescence microscope. RPE: retinal pigmented epithelium ; ONL: outer nuclear layer; INL: inner nuclear layer; GCL: ganglion cell layer.

FIG. 2. Nonhuman primate model: Live fluorescent retinal imaging at different time intervals (14, 21, 35, and 60 days p.i.) in Mac1 and Mac2. Both individuals received rAAV-2/4. CMV.gfp . (★) retinal detachment created by the subretinal injection.

FIG. 3. Nonhuman primate model: Two months p.i., neuroretina (A, B and D) and choroid/RPE (C) flatmounts were performed and examined under inverted fluorescence microscope. M, macula; ONH, optical nerve head, RV, retinal vessel.

FIG. 4. Nonhuman primate model: Sections from neuroretina (A, B) and choroid/RPE (C, D) flatmounts and were either analyzed by normal light microscope (A, C) or inverted fluorescence microscope (B, D). See legend FIG. 1 for RPE, ONL, INL and GCL.

FIG. 5. Vector shedding after subretinal delivery of rAAV-2/4. CMV.gfp in nonhuman primate (Mac1). PCR assay for sensitivity (A). Serum (s), lacrymal (I) and nasal (n) samples are represented (B). DNA marker (M), positive control on 25 pg of vector plasmid (+), negative control on water (−). Samples were collected 15 min, 2 hr and from day 1 to 28 p.i.

### DETAILED DESCRIPTION OF THE PREFERRED EMBODIMENTS

Throughout this application, several words are employed, the meaning of which should be understood according to the following definitions:

In the field of gene transfer, the term “vector” usually designates either a particle (viral or non-viral) comprising genetic material to be transferred into a host cell, or the vector genome itself (plasmid or recombinant viral vector, or any kind of DNA or RNA molecule). Throughout this specification, and for clarification purpose, the term “vector particle” will be used to designate the physical particle, including at least a nucleic acid molecule and a proteic moiety like a capsid, whereas the “vector genome” will designate the nucleic acid construct to be transferred. When neither one nor the other term is specified, both can be understood, depending on the context.

A frequent distinction is made between the “viral vectors”, which are directly derived from natural viruses, and “non-viral vectors”, which are principally made of synthetic molecules. However, in order to decrease the immunogenicity and toxicity of the first ones, and improve the efficiency of the latter, a growing number of chimeric vectors, partly made of synthetic molecules and partly of viral elements, are engineered. In this application, the distinction will me made as follows: a vector will be considered as a viral vector if it can be produced in cultured cells, whatever the number of integrated DNA constructs and helper plasmids, proteins, viruses etc. needed, and it will be considered as a non-viral vector in the opposite case, whatever the amount of viral elements included therein.

A “native AAV capsid” designates a capsid which is identical to the capsid of a natural AAV particle, by contrast with a “chimeric AAV capsid”, which is a capsid having a structure similar to that of a natural AAV, but with a few changes, such as, for example, VP1, VP2 and VP3 coming from more than one single AAV serotype (like the AAV-1/2 chimeric vectors described by Hauck et al, 2003), or such as a capsid made of an AAV capsid protein in which one or several amino acid have been deleted, added or modified. For example, it is possible to derive a cap gene from the cap gene of AAV-2 by replacing part of it with a sequence from the cap gene of AAV-4, in such a way that the expressed proteins are able to form a capsid which, in some aspects (for example, toxicity, immunogenicity, or stability) resembles the AAV-2 capsid and, in some others (for example, the specific targeting of RPE cells), resembles the AAV-4 capsid. Such a chimeric capsid, made of chimeric AAV-2/AAV-4 proteins, or of a mixture of proteins from AAV-2 and AAV-4, and retaining the tropism of AAV-4, will be qualified thereafter as a “chimeric AAV-4 capsid”.

The vector particles described in the present text are characterized by the fact that they “exhibit an AAV-4 capsid protein”. This means that at least one of VP1, VP2 and VP3 of AAV-4 are part of the vector particle, in such a way that they are exposed at its surface, thereby enabling the transduction of RPE cells. The AAV-4 capsid protein can be integrated into that particle (for example, in the case of a native or chimeric AAV capsid), or simply bound to the particle, by any physical means (for example, in the case of a non-viral vector).

The phrase “hybrid AAV vector particle”, equivalent to “AAV hybrid vector”, herein designates a vector particle comprising a native or chimeric AAV capsid including an rAAV vector genome and AAV Rep proteins, wherein Cap, Rep and the ITRs of the vector genome come from at least 2 different AAV serotypes. These serotypes can be indicated. Examples of such hybrid vectors are rAAV-2/4 hybrid vectors, sometimes merely referred to as rAAV-2/4 vectors, comprising an AAV-4 capsid and a rAAV genome with AAV-2 ITRs, with a Rep protein from either AAV-2 (Rabinowitz, Rolling et al. 2002) or AAV-4 (Kaludov, Brown et al. 2001).

Throughout this application, the terms “gene transfer” and “transduction” will be indifferently used to express the fact that a nucleic acid sequence enters a cell, no matter its later fate in said cell. Therefore, if the vector genome enters the cell, the cell is transduced, even if the transgene is not expressed. The integration of the vector genome into the cell genome or not is also not to be taken in consideration to determine whether the cell is transduced.

The word “transgene” herein designates any nucleotide sequence coding for any polypeptide, structural protein, enzyme etc., the expression of which is wanted in a target cell, for any kind of reason. It can also designate a non-coding sequence, for example an antisense sequence or the sequence of an interferent RNA aimed at decreasing the expression of a gene, or even a sequence which will be transcribed into a ribozyme. The expression “gene of interest” can also be used in place of “transgene”.

As described in the examples below, the inventors have showed that the type −4 AAV capsid allows exclusive and stable transduction of RPE cells after subretinal delivery, at least in rAAV-2/4 hybrid vectors with a CMV-driven transgene. This is a unique feature in the nonhuman primate model. None of the other rAAV serotypes provided such unambiguous specificity.

A first aspect of the present invention is hence a method for selectively transducing retinal pigment epithelium (RPE) cells in an eye of a mammal, comprising administering to said mammal a vector particle exhibiting an AAV-4 capsid protein. According to this method, the administration of the vector particle is preferably performed by subretinal delivery.

The AAV capsid is composed of three related proteins, VP1, VP2 and VP3 of decreasing size, present at a ration of about 1:1:10, respectively, and derived from a single cap gene by alternative splicing and alternative start codon usage. No other protein is exposed at the surface of the AAV vectors used in the experiments described below, which implies that the observed tropism specificity is indeed due to the AAV-4 capsid protein. Therefore, any vector particle, viral or non viral, exhibiting the AAV-4 capsid protein, will most probably present the same tropism as those described below.

According to the invention, the vector particle preferably comprises a recombinant AAV genome comprising a sequence of interest flanked by AAV ITRs. It has been demonstrated that it is possible to encapsidate in an AAV-4 capsid a recombinant AAV genome having the AAV-2 ITRs (Kaludov, Brown et al. 2001). Since AAV-2 ITRs-flanking vectors are, so far, the most characterized in preclinical and clinical trials, the use of such an rAAV-2/4 hybrid particle vector is presently advantageous. However, this is not compulsory, and other sequences can be used in the genome of the vectors according to the present invention. For example, AAV-4 ITRs can be used in the vectors according to the present invention, as well as ITRs from other serotypes. As mentioned above, rAAV-2/4 vectors with an AAV-4 capsid and AAV-2 ITRs can be obtained with a Rep protein from either AAV-2 (Rabinowitz, Rolling et al. 2002) or AAV-4 (Kaludov, Brown et al. 2001).

Retinal degenerative diseases such as retinal macular degeneration or retinitis pigmentosa constitute a broad group of diseases that all share one critical feature, the progressive loss of cells in the retina. There is currently no effective treatment available by which the course of these disorders can be modified and visual dysfunction eventually progresses to total blindness. Gene therapy represents a possible approach to treating retinal degenerations because the eye is easily accessible and allows local application of therapeutic vectors with reduced risk of systemic effects. Furthermore, transgene expression within the retina and effects of treatments may be monitored by a variety of non-invasive examinations.

Therefore, another object of the present invention is a method for preventing, treating or alleviating an eye disease in a mammal, by delivering into the eye of said mammal a vector particle exhibiting an AAV-4 capsid protein and comprising a vector genome encoding a transgene which, when expressed in RPE cells, has a beneficial effect on said eye disease. As mentioned above, the vector particle is preferably administered by subretinal delivery.

Using the methods and gene delivery vectors provided herein, a wide variety of diseases of the eye may be readily treated or prevented, including for example, inherited or non-inherited retinal degenerations, retinal dystrophies, retinitis pigmentosa, macular degenerations, Leber's congenital amaurosis (LCA), cone-rod dystrophies, neovascular diseases of the eye, choroidal degenerations, choroidal sclerosis, diabetic retinopathies, proliferative vitreoretinopathies, choroïderemia, glaucoma and metabolic disorders such as Sly syndrome (MPS VII, due to a defect in the beta-glucoronidase gene) and gyrate atrophy (due to a defect in the ornithine-delta-aminotransferase gene, OAT), retinal detachment or injury and retinopathies (whether inherited, induced by surgery, trauma, a toxic compound or agent, or photically induced).

Gene therapy of the eye with vectors according to the present invention can be performed either by introducing in RPE cells a functional copy of a gene that is deficient therein (gene replacement therapy), or by delivering to RPE cells a gene which will have a beneficial effect on the eye disease to be treated (symptomatic therapy).

Examples of genes that can be used for gene replacement therapy are genes that are specifically expressed in RPE cells, such as RGR (Retinitis pigmentosa, RP, chromosome 10), RDH5 (fundus albipunctatus, chr. 12), RPE65 (Leber's congenital amaurosis, LCA, chr. 1), RLBPL (RP, chr. 15), MERTK (RP, chr. 2), LRAT (RP, chr. 4), REP1 (choroïdemia, Xp2l), RBP4 (RPE degeneration, chr. 10) and usherin (Usher syndrome type 2A), or genes that are also expressed in other cell-types, such as Myo7A (Usher syndrome type 1), ELOVL4 (macular degeneration, chr. 6), EFEMPI (Malattia Leventinese disease, chr. 15), VMD2 (Best Disease, chr. 11), TIMP3 (Sorsby's fundus dystrophy, chr. 22), AIPL1 (LCA, chr. 7), and CRB1 (RP, chr. 1).

Examples of genes that can be used for symptomatic therapy are trophic factors (such as neurotrophic factors or survival factors), growth factors, anti-angiogenic factors, survival factors, suicide genes, anti-apoptotic factors, and some enzymes. Representative examples of neurotrophic factors include NGF, BDNF, CNTF, NT-3, NT-4, FGF-2, FGF-5, FGF-18, FGF-20 and FGF-21. An example of growth factor is bFGF. Representative examples of anti-angiogenic factors include PEDF, TIMP3, EGF, endostatin, soluble Flt-1, and soluble Tie-2 receptor. A particular survival factor which can be used in the methods described herein is the rod-derived survival/viability factor (rdcvf, described in WO 02/081513). A suicide gene can be that of HSV-1 thymidine kinase. Caspase inhibitors can be cited as anti-apoptotic factors. Relevant enzymes include β-glucuronidase, neuraminidase, sphingomyelinase, sulfatases, arylsulfatase β, α-neuraminidase, gangliosidase, tripeptidyl protease, CLN3 and palmitoyl protein thioesterase (PPT).

Within certain embodiments of the invention, the gene delivery vector is used to deliver and express an anti-angiogenic factor for the treatment, prevention, or inhibition of diabetic retinopathy, wet ARMD, and other neovascular diseases of the eye (e.g., ROP). Within other embodiments it is desirable that the gene delivery vector be used to deliver and express a neurotrophic growth factor to treat, prevent, or inhibit diseases of the eye, such as, for example, glaucoma, retinitis pigmentosa, and dry ARMD.

In the methods according to the present invention, the transgene can hence be selected (without being limitative) in the group consisting of a ribozyme, an antisense RNA, an interferent RNA, and a sequence encoding a polypeptide or protein selected in the group consisting of RGR, RDH5, RPE65, RLBP1, MERTK, LRAT, REP1, RBP4, usherin, Myo7A, TIMP3, ELOVL4, AIPL1, CRB1, trophic factors such as neurotrophic factors including NGF, BDNF, CNTF, NT-3, NT-4, FGF-2, FGF-5, FGF-18, FGF-20 and FGF-21, growth factors like bFGF, anti-angiogenic factors such as PEDF, TIMP3, EGF, endostatin, soluble Flt-1, and soluble Tie-2 receptor, survival factors like the rod-derived survival/viability factor (rdcvf, described in WO 02/081513), suicide genes like HSV-1 thymidine kinase, anti-apoptotic factors such as caspase inhibitors, and enzymes selected in the group including β-glucuronidase, neuraminidase, sphingomyelinase, sulfatases, arylsulfatase β, α-neuraminidase, gangliosidase, tripeptidyl protease, CLN3 and palmitoyl protein thioesterase (PPT).

In these vectors, the transgene is preferably operably linked to a promoter, which can be a constitutive promoter such as the CMV promoter used in the examples, a light-switchable—also called photo-activated—promoter (e.g., the PER1 promoter of the period gene in drosophila, or a promoter driving the expression of a phytochrome of Arabidopsis), or a regulated promoter (e.g., “tet” promoters, the ecdysome system, or other systems of regulation), a viral promoter (e.g., the CMV or RSV promoters), a tissue or cell-specific promoter (e.g., a rod, cone, or ganglia-derived promoter), or a rhodopsin promoter.

Due to the specific tropism of the vector particles exhibiting an AAV-4 capsid protein, the above method can be performed according to the invention, when the disease is due to a deficiency in retinal pigment epithelium (RPE) cells. This is the case for example of Leber congenital amaurosis (RPE65) or retinitis pigmentosa due to a mutated mertk gene.

In a method according to the invention, to treat, prevent or alleviate diseases associated with the EFEMPI, VMD2, TIMP3 and ELOVL4 genes, the transgene can for example be an antisense, an interferent RNA or a ribozyme to inhibit the expression of the mutated gene.

Alternatively, the methods according to the invention can be used to prevent, treat or alleviate a disease which is due to a deficiency in the photoreceptors. This is possible for example if the vector particles transduce retinal pigment epithelium (RPE) cells and express therein a neurotrophic factor or a survival factor, which will diffuse to the photoreceptors.

Within yet other embodiments, it may be desirable to use either a gene delivery vector which expresses both an anti-angiogenic molecule and a neurotrophic growth factor, or two separate vectors which independently express such factors, in the treatment, prevention, or inhibition of an eye disease (e.g., for diabetic retinopathy).

Within further embodiments of the invention, the above-mentioned methods utilizing gene delivery vectors may be administered along with other methods to or therapeutic regimens, including for example, photodynamic therapy (e.g., for wet ARMD), laser photocoagulation (e.g., for diabetic retinopathy and wet ARMD), and intraocular pressure reducing drugs (e.g., for glaucoma).

Another object of the present invention is a vector particle which exhibits an AAV-4 capsid protein, and comprises a vector genome encoding a transgene which, when specifically expressed in retinal pigment epithelium (RPE) cells, can treat, prevent or alleviate the effects of an eye disease. This transgene can be for example selected in the group of RGR, RDH5, RPE65, RLBP1, MERTK, LRAT, REP1, RBP4, usherin, Myo7A, ELOVL4, EFEMPI, VMD2, AIPL1, CRB1, TIMP3 and.

The present invention also pertains to a pharmaceutical composition for administration in the eye, comprising vector particles according to the invention (as described above), and a pharmaceutically acceptable carrier.

Another aspect of the invention is the use of a vector particle as described above, for the manufacture of a composition for treating a disease in the eye or preventing cell damage in the eye.

While the invention has been described in terms of various preferred embodiments, the skilled artisan will appreciate that various modifications, substitutions, omissions and changes may be made without departing from the scope thereof. Accordingly, it is intended that the scope of the present invention be limited by the scope of the following claims, including equivalents thereof.

Other characteristics of the invention will also become apparent in the course of the description which follows of the biological assays which have been performed in the framework of the invention and which provide it with the required experimental support, without limiting its scope.

### EXAMPLES

The experiments described below have been performed using the following materials and methods:

### rAAV-214 Vectors

Recombinant AAV-2/4 vectors carried a CMV.gfp genome flanked by AAV-2 ITRs encapsidated in an AAV-4 shell. rAAV-2/4 vectors were produced as previously described (Kaludov, Brown et al. 2001), by cotransfection of a plasmid carrying the rep and cap AAV-4 genes having the sequence described in WO 98/11244, the pXX6 helper plasmid described before (Xiao, Li et al. 1998), and the SSVgfp plasmid carrying the recombinant vector (Rolling, Shen et al. 1999). The rAAV titer was determined by dot blot and expressed as vector genome/ml (vg/ml) (Salvetti, Oreve et al. 1998). It was 4×10¹² vg/ml for rAAV-2/4.

### Subretinal Injection

In rat, anesthesia, surgical procedures and post surgery care were performed as described previously in (Duisit, Conrath et al. 2002). Primates were purchased BioPrim, Baziège, France. All animals were cared for in accordance with the ARVO statement for the use of animals in ophthalmic and vision research. Subretinal injections were performed via a transvitreal approach under isofluorane gas anesthesia. A vitrectomy was performed in the two macaques—Mac1 and Mac2—before the subretinal injection of 40 μl and 120 μl of rAAV-2/4. CMV.gfp, respectively.

In vivo GFP fluorescence Imaging, Retina Flatmounting, and Tissue Sections.

GFP protein expression in live rats and primates was monitored at weekly intervals by fluorescent retinal imaging as described in (Duisit, Conrath et al. 2002). The sclera/choroid/RPE and neuroretina flatmounting was performed on 4% paraformaldehyde-fixed enucleated eyes as previously described (Duisit, Conrath et al. 2002). Tissue sections were also made. For macaques eyes, RPE-choroid layers were separated from the sclera.

Detection of rAAV Vector DNA in Body Fluids After Subretinal Delivery in Macaques.

Biological samples and PCR analysis were processed as previously described (Favre, Provost et al. 2001). The 5′ primer (5′-AAGTTCATCTGCACCACCG-3′) and the 3′ primer (5′-TGTTCTGCTGGTAGTGGTCG-3′) were both located in the gfp DNA sequence. PCR-amplified vector sequence yielded a 424-bp fragment. After initial denaturation at 95° C. for 5 min, 40 cycles were run at 94° C. for 30 s, 60° C. for 30 s, 72° C. for 30 s, followed by incubation at 72° C. for 10 min using Taq DNA polymerase (Promega) in a Perkin-Elmer thermocycler (PE, USA). Amplified products were analyzed by agarose gel electrophoresis.

### Example 1

### Subretinal Delivery of rAAV-2/4. CMV.qfp in Rats

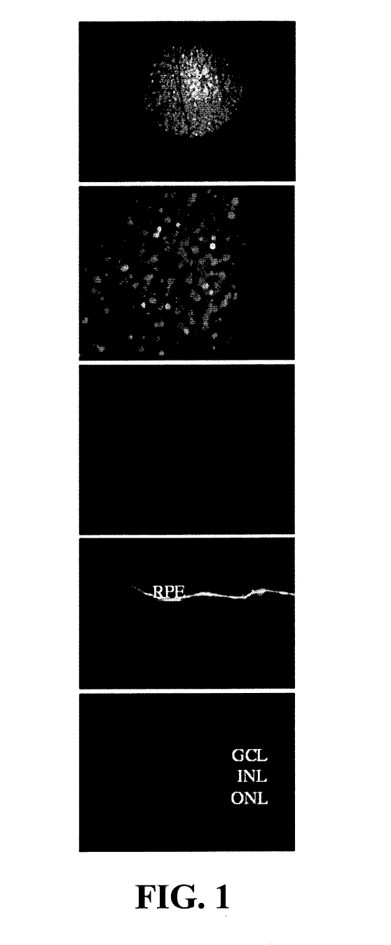

Subretinal injection of rAAV-2/4. CMV.gfp (4×10¹² vg/ml corresponding to 8×10⁹ vg/injection) were performed on three Wistar rats. In retina flatmounts, using a fluorescence inverted microscope, rAAV-2/4-mediated gene expression was restricted to the sclera/choroid/RPE layer (FIG. 1B) and more specifically to RPE cells (FIG. 1D). No signal was ever detected in the neuroretina layer (FIG. 1C and E).

### Example 2

### Subretinal Delivery of rAAV-2/4. CMV.qfp in Nonhuman Primates

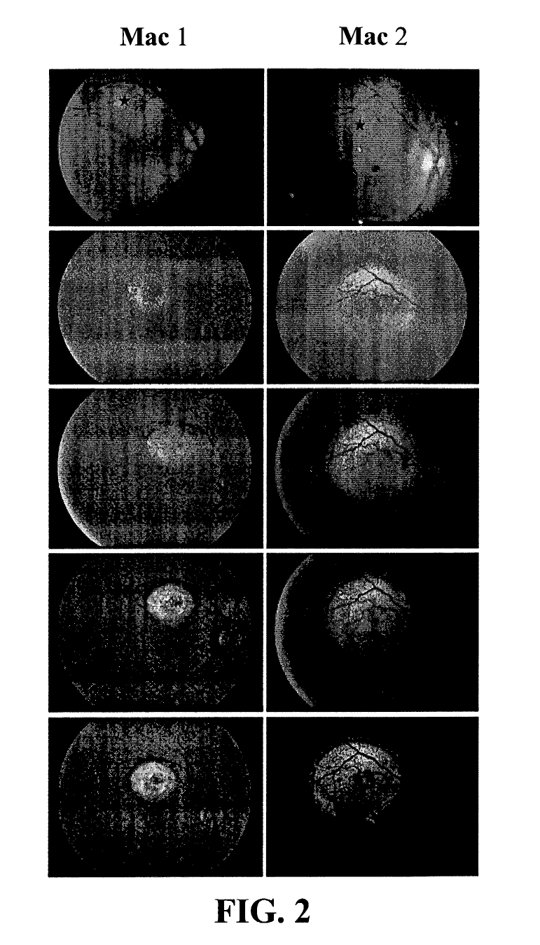

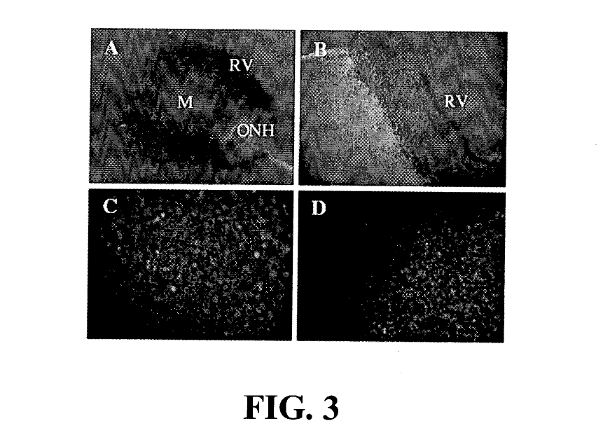

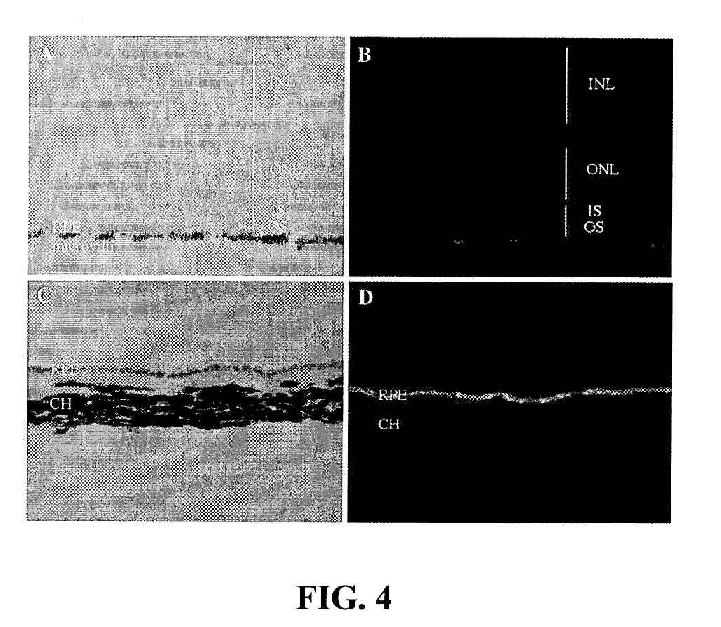

To test the tropism of the rAAV-2/4 vector in a relevant preclinical animal model, subretinal injection of 40 μl and 120 μl of rAAV-2/4. CMV.gfp was performed via a transvitreal route in Mac1 and Mac2 resulting in retinal detachment outside and within the macula, respectively (FIG. 2). The rAAV-2/4 vector resulted in a detectable GFP signal (14 days p.i. in both animals with a maximum expression level ≅60 days p.i. (FIG. 2). While the GFP signal was homogeneous over the targeted area in Mac 1, Mac2 displayed a less intense GFP signal within the macula. This result is not surprising since in primates, RPE cells are strongly pigmented resulting in partial fluorescence quenching. Retinal flatmounts were obtained from Mac2 sixty-five days p.i. During the dissection, the choroïd/RPE layer was separated from the neuroretina. However, during this process, pigmented RPE cells located between the two vascular arcades did not detach easily from the neuroretina as evidenced by the presence of RPE pigmentation on the neuroretina (FIG. 3 A and B). This technical handicap was also observed on non-injected primate eyes and, therefore, was not attributed to the vector or the injection itself (data not shown). As a consequence, GFP signal was observed on both choroid/RPE and neuroretina flatmounts (FIG. 3C and D). However, the GFP signal clearly displayed the typical hexagonal shape of RPE cells on both flatmounts. No fine pixelized signal was found in the neuroretina suggesting that only RPE cells were actually transduced. The signal observed in the neuroretina flatmount could be due either to entire tranduced RPE cells or transduced RPE cell microvilli still attached to the photoreceptor outer segments, or both. In support of this, neuroretina sections displayed a pigmented top layer that corresponded to residual attached RPE microvilli (FIG. 4A). The only detectable fluorescence signal in the top layer of the neuroretina were transduced RPE microvilli (FIG. 4B) and no GFP signal could be detected in the outer nuclear layer or in photoreceptor outer segments layer. Furthermore, RPE cells were expressing GFP in the choroid/RPE layer (FIG. 4C and D). These results demonstrated that an accurate subretinal injection of rAAV-2/4 vector in macaques lead to unique and exclusive transduction of RPE cells.

### Example 3

### Vector Shedding After Subretinal Delivery of rAAV in Nonhuman Primate

To provide additional preclinical data from large animal models, the inventors have looked for vector shedding after rAAV delivery in the subretinal space of Mac1 and Mac2 primates. PCR was used to detect rAAV vector genome in several body fluids (Table 1).

TABLE 1
detection of rAAV vector sequences by PCR in body fluids.

| | serum | lacrymal | nasal | urine | feces |
|---|---|---|---|---|---|
| MAC1 | 2 h-16 d | 15′-2 h | 15′-2 h | negative | negative |
| MAC2 | negative | 15′-2 h | negative | negative | negative |

D, days post infection, h, hours pi., and ′, minutes p.i.

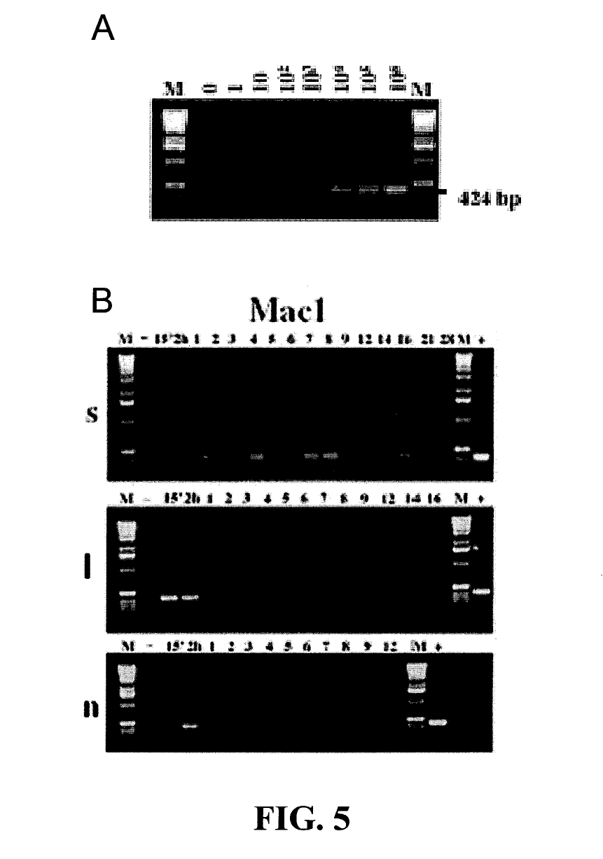

The sensitivity of the assay was first evaluated by incubating a known number of viral particles with saline before extracting the DNA as described (Favre, Provost et al. 2001). The results indicated that a threshold of 10³ to 10⁴ vg particles could be detected (FIG. 5A). Serum, lacrymal, and nasal samples were collected from 15 min to 2 months p.i. and analyzed by PCR to detect the gfp DNA. Vector DNA could be detected in the serum as soon as 15 minutes after rAAV administration and up to 25 days in some instances (FIG. 5B, and Table 1). For lacrymal and nasal fluids, viral genome was also detected as soon as 15 minutes and up to 4 days (FIG. 5B, and Table 1). Overall, the important finding is that rAAV vector was shed in various biological fluids in animals using a clinically relevant surgical procedure and an accurate subretinal delivery (Table 1).

### Discussion

This study showed that the type −4 AAV capsid allows exclusive and stable transduction of RPE cells after subretinal delivery, at least with a CMV-driven transgene. This is a unique feature in the nonhuman primate model. None of the other rAAV serotypes provided such unambiguous specificity. Therefore, rAAV-2/4 represents an important candidate vector for therapy of RPE-specific genetic diseases such as retinitis pigmentosa due to a mutated mertk gene (Gal, Li et al. 2000) and Leber congenital amaurosis (Gu, Thompson et al. 1997; Acland, Aguirre et al. 2001). In the mouse central nervous system, rAAV-4 was also found to restrict transgene expression to the ependymal cells (Davidson, Stein et al. 2000). This suggests that RPE and ependymal cells may share a common receptor and/or coreceptor.

Maximal transgene expression occured ≅60 days p.i. in both macaques. This pattern is also shared by the (non chimeric) AAV-2 vector, suggesting that it is linked to the AAV-2 ITRs biology. This may be an advantage of the chimeric rAAV vectors over the non-chimeric ones since the AAV-2 ITRs-flanking vectors are, so far, the most characterized in preclinical and clinical trials.

Another important finding in the present study is the discovery of shedding of the rAAV vector immediately and up to several weeks after delivery using a clinically relevant surgical approach. Shedding in lacrymal and nasal fluids were not unexpected because of the transvitreal approach used. However, it remains difficult to explain the so long presence of the vector in the serum of Mac1. Whether it is possible that non infectious rAAV vectors trapped in the subretinal space are slowly released to the highly vascularized choroïd remains to be investigated. This finding is in contrast to intramuscular delivery of rAAV-2 vectors where detection in the sera was for only two days p.i. in factor IX deficient patients (Kay, Manno et al. 2000), and for 6 days in non human primates (Favre, Provost et al. 2001).

Overall, this study provides evidence that exclusive targeting of the RPE cells is now possible using the rAAV-4 serotype in the primate, making it an optimal candidate for future clinical trials for Leber congenital amaurosis.

### References

Acland, G. M., G. D. Aguirre, et al. (2001). “Gene therapy restores vision in a canine model of childhood blindness.” Nat Genet 28(1): 92-5.

Ali, R. R., M. B. Reichel, et al. (1998). “Adeno-associated virus gene transfer to mouse retina.” Hum Gene Ther 9(1): 81-6.

Ali, R. R., M. B. Reichel, et al. (1996). “Gene transfer into the mouse retina mediated by an adeno-associated viral vector.” Hum Mol Genet 5(5): 591-4.

Ali, R. R., G. M. Sarra, et al. (2000). “Restoration of photoreceptor ultrastructure and function in retinal degeneration slow mice by gene therapy.” Nat Genet 25(3): 306-10.

Auricchio, A., G. Kobinger, et al. (2001). “Exchange of surface proteins impacts on viral vector cellular specificity and transduction characteristics: the retina as a model.” Hum Mol Genet 10(26): 3075-81.

Bennett, J., A. M. Maguire, et al. (1999). “Stable transgene expression in rod photoreceptors after recombinant adeno-associated virus-mediated gene transfer to monkey retina.” Proc Natl Acad Sci USA 96(17): 9920-5.

Davidson, B. L., C. S. Stein, et al. (2000). “Recombinant adeno-associated virus type 2, 4, and 5 vectors: transduction of variant cell types and regions in the mammalian central nervous system.” Proc Natl Acad Sci USA 97(7): 3428-32.

Duisit, G., H. Conrath, et al. (2002). “Five recombinant simian immunodeficiency virus pseudotypes lead to exclusive transduction of retinal pigmented epithelium in rat.” Mol Ther 6(4): 446-54.

Favre, D., N. Provost, et al. (2001). “Immediate and long-term safety of recombinant adeno-associated virus injection into the nonhuman primate muscle.” Mol Ther 4(6): 559-66.

Gal, A., Y. Li, et al. (2000). “Mutations in MERTK, the human orthologue of the RCS rat retinal dystrophy gene, cause retinitis pigmentosa.” Nat Genet 26(3): 270-1.

Green, E. S., K. G. Rendahl, et al. (2001). “Two animal models of retinal degeneration are rescued by recombinant adeno-associated virus-mediated production of FGF-5 and FGF-18. ” Mol Ther 3(4): 507-15.

Gu, S. M., D. A. Thompson, et al. (1997). “Mutations in RPE65 cause autosomal recessive childhood-onset severe retinal dystrophy.” Nat Genet 17(2): 194-7.

Hauck, B., L. Chen, et al. (2003). “Generation and characterization of chimeric recombinant AAV vectors.” Molecular therapy 7(3): 419-25.

Kaludov, N., K. E. Brown, et al. (2001). “Adeno-associated virus serotype 4 (AAV-4) and AAV-5 both require sialic acid binding for hemagglutination and efficient transduction but differ in sialic acid linkage specificity.” J Virol 75(15): 6884-93.

Kay, M. A., C. S. Manno, et al. (2000). “Evidence for gene transfer and expression of factor IX in haemophilia B patients treated with an AAV vector.” Nat Genet 24(3): 257-61.

Lau, D., L. H. McGee, et al. (2000). “Retinal degeneration is slowed in transgenic rats by AAV-mediated delivery of FGF-2.” Invest Ophthalmol Vis Sci 41(11): 3622-33.

LaVail, M. M., D. Yasumura, et al. (2000). “Ribozyme rescue of photoreceptor cells in P23H transgenic rats: long-term survival and late-stage therapy.” Proc Natl Acad Sci USA 97(21): 11488-93.

Liang, F. Q., N. S. Dejneka, et al. (2001). “AAV-mediated delivery of ciliary neurotrophic factor prolongs photoreceptor survival in the rhodopsin knockout mouse.” Mol Ther 3(2): 241-8.

Rabinowitz, J. E., F. Rolling, et al. (2002). “Cross-packaging of a single adeno-associated virus (AAV) type 2 vector genome into multiple AAV serotypes enables transduction with broad specificity.” J Virol 76(2): 791-801.

Rolling, F., W. Y. Shen, et al. (1999). “Evaluation of adeno-associated virus-mediated gene transfer into the rat retina by clinical fluorescence photography.” Hum Gene Ther 10(4): 641-8.

Salvetti, A., S. Oreve, et al. (1998). “Factors influencing recombinant adeno-associated virus production.” Hum Gene Ther 9(5): 695-706.

Xiao, X., J. Li, et al. (1998). “Production of high-titer recombinant adeno-associated virus vectors in the absence of helper adenovirus.” J Virol 72(3): 2224-32.

Yang, G. S., M. Schmidt, et al. (2002). “Virus-mediated transduction of murine retina with adeno-associated virus: effects of viral capsid and genome size.” J Virol 76(15): 7651-60.

1. A method for selectively transducing retinal pigment epithelium (RPE) cells in an eye of a mammal, comprising administering to said mammal a vector particle exhibiting an AAV-4 capsid protein.

2. The method of claim 1, wherein the administration of the vector particle is performed by subretinal delivery.

3. The method of claim 1, wherein said vector particle comprises a native or a chimeric AAV capsid and a vector genome.

4. The method of claim 1, wherein the vector particle comprises a recombinant AAV genome comprising a sequence of interest flanked by AAV ITRs.

5. The method of claim 4, wherein the AAV ITRs are the AAV-2 ITRS.

6. The method of claim 4, wherein the AAV ITRs are the AAV-4 ITRs.

7. A method for preventing, treating or alleviating an eye disease in a mammal, by delivering into the eye of said mammal a vector particle exhibiting an AAV-4 capsid protein and comprising a vector genome encoding a transgene which, when expressed in RPE cells, has a beneficial effect on said eye disease.

8. The method of claim 7, wherein the administration of the vector particle is performed by subretinal delivery.

9. The method of claim 7, wherein said eye disease is an inherited or non-inherited retinal degeneration, a retinal dystrophy, a retinitis pigmentosa, a macular degeneration, a Leber's congenital amaurosis, a cone-rod dystrophy, a neovascular disease of the eye, a choroidal degeneration, a choroidal sclerosis, a diabetic retinopathy, a proliferative vitreoretinopathy, a choroïderemia, a glaucoma, a metabolic disorder, a Sly syndrome, a gyrate atrophy, a retinal detachment or injury, or a retinopathy.

10. The method of claim 7, wherein said transgene is selected in the group consisting of a ribozyme, an antisense RNA, an interferent RNA, and a sequence encoding a polypeptide or protein selected in the group consisting of RGR, RDH5, RPE65, RLBP1, MERTK, LRAT, REP1, RBP4, usherin, Myo7A, TIMP3, ELOVL4, EFEMPI, VMD2, AIPL1, CRB1, neurotrophic factors including NGF, BDNF, CNTF, NT-3, NT-4, FGF-2, FGF-5, FGF-18, FGF-20 and FGF-21, growth factors including bFGF, anti-angiogenic factors including PEDF, TIMP3, EGF, endostatin, soluble Flt-1, and soluble Tie-2 receptor, survival factors including the rod-derived survival/viability factor, suicide genes including HSV-1 thymidine kinase, anti-apoptotic factors including caspase inhibitors, and enzymes including β-glucuronidase, neuraminidase, sphingomyelinase, sulfatases, arylsulfatase β, α-neuraminidase, gangliosidase, tripeptidyl protease, CLN3 and palmitoyl protein thioesterase (PPT).

11. The method of claim 7, wherein said transgene is operably linked to a constitutive promoter, a regulated promoter, a photo-activated promoter, a viral promoter, a cell-specific promoter or a rhodopsin promoter.

12. The method of claim 7, wherein said disease is due to a deficiency in retinal pigment epithelium (RPE) cells.

13. The method of claim 12, wherein said disease is due to a mutated EFEMPI, VMD2, TIMP3 or ELOVL4 gene.

14. The method of claim 13, wherein the transgene is selected in the group consisting of an antisense, an interferent RNA or a ribozyme to inhibit the expression of the mutated EFEMPI, VMD2, TIMP3 or ELOVL4 gene.

15. The method of claim 7, wherein said disease is due to a deficiency in the photoreceptors.

16. The method of claim 15, wherein the vector particle transduces retinal pigment epithelium (RPE) cells and expresses therein a neurotrophic factor or a survival factor which is able to diffuse into the photoreceptors.

17. A vector particle which exhibits an AAV-4 capsid protein, and comprises a vector genome encoding a transgene which, when specifically expressed in retinal pigment epithelium (RPE) cells, can treat, prevent or alleviate the effects of an eye disease.

18. The vector particle of claim 17, wherein the transgene is selected in the group of RGR, RDH5, RPE65, RLBP1, MERTK, LRAT, REP1, RBP4 and usherin.

19. The vector particle of claim 17, wherein said vector particle comprises a native or a chimeric AAV capsid.

20. The vector particle of claim 17, wherein the vector particle comprises a recombinant AAV genome comprising a sequence of interest flanked by AAV ITRs.

21. The vector particle of claim 20, wherein the AAV ITRs are the AAV-2 ITRs.

22. A pharmaceutical composition for administration in the eye, comprising vector particles according to any of claims 17 to 21, and a pharmaceutically acceptable carrier.
